(19) United States
(12) Patent Application Publication
(10) Pub. No.: US 2009/0270754 A1
(43) Pub. Date: Oct. 29, 2009

(54) Control Apparatus, Control Method, Computer Program for the Control Method, and Recording Medium Having Recorded Therein the Computer Program for the Control Method

(76) Inventor: Tomohisa MORIDAIRA, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/428,093

(22) Filed: Apr. 22, 2009

(30) Foreign Application Priority Data
Apr. 24, 2008 (JP) P2008-113797

(51) Int. Cl. A61B 5/0476

(52) U.S. Cl. 600/544

(57) ABSTRACT

A control apparatus includes: a brain-activity-signal processing unit that processes plural brain activity signals and detects plural estimation values for estimating brain activity; and a control unit that controls a control target on the basis of the estimation values of the brain activity. The control unit switches a control target region of the control target according to at least one of the plural brain activity signals and controls the switched control target region according to the plural brain activity signals.

### BACKGROUND OF THE INVENTION

1. Field of the Invention

The present invention relates to a control apparatus, a control method, a computer program for the control method, and a recording medium having recorded therein the computer program for the control method and can be applied to, for example, various kinds of control by a Brain Computer Interface (BCI) and various kinds of control by a Brain Machine Interface (BMI). The present invention switches a control target region according to an estimation value used for control and controls the switched control target region according to the estimation value used for the control to thereby make it possible to efficiently use a signal obtained by brain measurement for the various kinds of control.

2. Description of the Related Art

In recent years, the advance of brain function measuring methods employing the functional Near Infrared Spectroscopy (fNIRS), the functional Magnetic Resonance Imaging (fMRI) method, the Electro Encephalo Graphy (EEG) method, the Magneton Encephalo Graphy (MEG) method, and the like make it possible to apply brain measurement to various functional local regions. As a result, detection of a highly independent signal is becoming possible.

Concerning the brain measurement, JP-A-2006-95266 proposes a method of measuring, with the fNIRS method, hemoglobin concentration in a brain blood flow or a change in the hemoglobin concentration using near infrared light and estimating a brain activity state of a person on the basis of a result of the measurement.

### SUMMARY OF THE INVENTION

A signal obtained by the brain measurement can be arbitrarily created on a human side through execution of training that makes use of plasticity of a brain. Therefore, if a control apparatus is established by efficiently using the signal obtained by the brain measurement, it is considered to be possible to provide a more highly convenient user interface compared with that in the past.

Therefore, it is desirable to obtain a control apparatus, a control method, a computer program for the control method, and a recording medium having recorded therein the computer program for the control method that can efficiently use a signal obtained by brain measurement for various kinds of control.

According to an embodiment of the present invention, there is provided a control apparatus including: a brain-activity-signal processing unit that processes plural brain activity signals and detects plural estimation values for estimating brain activity; and a control unit that controls a control target on the basis of the estimation values of the brain activity, wherein the control unit switches a control target region of the control target according to at least one of the plural brain activity signals and controls the switched control target region according to the plural brain activity signals.

According to another embodiment of the present invention, there is provided a control method including the steps of: processing plural brain activity signals and detecting plural estimation values for estimating brain activity; and controlling a control target on the basis of the estimation values of the brain activity, wherein the control step includes the steps of: switching a control target region of the control target according to at least one of the plural brain activity signals; and controlling the switched control target region according to the plural brain activity signals.

According to still another embodiment of the present invention, there is provided a computer program for a control method executable by a computer, the computer program including the steps of: processing plural brain activity signals and detecting plural estimation values for estimating brain activity; and controlling a control target on the basis of the estimation values of the brain activity, wherein the control step includes the steps of: switching a control target region of the control target according to at least one of the plural brain activity signals; and controlling the switched control target region according to the plural brain activity signals.

According to still another embodiment of the present invention, there is provided a recording medium having recorded therein a computer program for a control method executable by a computer, the computer program for a control method including the steps of: processing plural brain activity signals and detecting plural estimation values for estimating brain activity; and controlling a control target on the basis of the estimation values of the brain activity, wherein the control step includes the steps of: switching a control target region of the control target according to at least one of the plural brain activity signals; and controlling the switched control target region according to the plural brain activity signals.

According to the embodiments of the present invention, since the control target region of the control target is switched by at least one of the plural brain activity signals and the switched control target region is controlled by the plural brain activity signals, it is possible to control a control target having a high degree of freedom compared with a degree of freedom of the estimation values. Therefore, it is possible to efficiently use a signal obtained by brain measurement for various kinds of control.

According to the embodiments of the present invention, it is possible to efficiently use a signal obtained by brain measurement for various kinds of control.

### BRIEF DESCRIPTION OF THE DRAWINGS

FIG. 1 is a flowchart of a processing procedure of a central processing unit in a control system according to a first embodiment of the present invention;

FIG. 2 is a block diagram of the control system according to the first embodiment;

FIG. 3 is a block diagram of a measuring apparatus in the control system shown in FIG. 2;

FIG. 4 is a plan view of a measuring device in the measuring apparatus shown in FIG. 3;

FIG. 5 is a diagram for explaining processing of various prior tasks;

FIG. 6 is a time chart of processing for measuring a brain activity signal when an emotion recall task is used;

FIG. 7 is a diagram of arithmetic processing for processing a brain activity signal;

FIG. 8 is a time chart for explaining measurement of a brain activity signal;

FIG. 9 is a diagram of arithmetic processing for processing of the brain activity signal obtained by the measurement shown in FIG. 8;

FIGS. 10A and 10B are plan views for explaining clustering;

FIG. 11 is a flowchart of prior processing;

FIG. 12 is a flowchart of estimation execution processing;

FIG. 13 is a flowchart for explaining control of a single agent;

FIG. 14 is a diagram for explaining a control target in a control system according to a second embodiment of the present invention; and

FIG. 15 is a flowchart of a processing procedure of a central processing unit in the control system according to the second embodiment.

### DESCRIPTION OF THE PREFERRED EMBODIMENTS

Embodiments of the present invention are explained in detail below with reference to the accompanying drawings as appropriate.

### First Embodiment

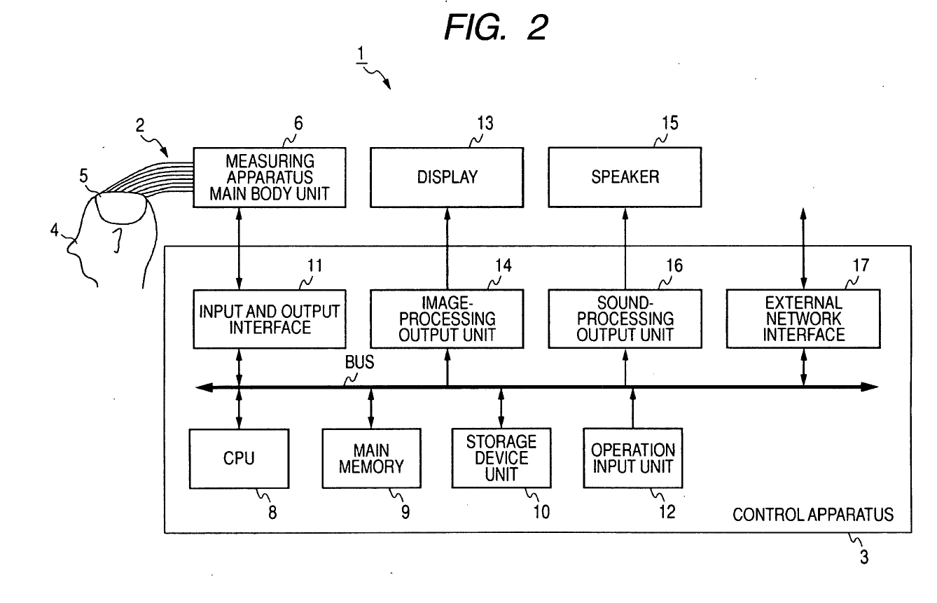

### (1) Overall Configuration of the Embodiment (FIGS. 2 to 4)

FIG. 2 is a block diagram of a control system according to a first embodiment of the present invention. A control system 1 controls a desired control target using a BCI.

In this control system 1, the measuring apparatus 2 operates according to the control by the control apparatus 3 and detects brain activity signals of a user 4 in plural measurement regions. The measuring apparatus 2 includes a measuring device 5 attached to the head of the user 4 and a measuring apparatus main body 6 that detects a brain activity signal via the measuring device 5. The measuring apparatus 2 emits a near infrared ray to a brain surface layer, receives emitted light to the outside of the head, which changes according to hemoglobin concentration in a blood flow or a change in the hemoglobin concentration, and detects a brain activity signal. Consequently, the measuring apparatus 2 detects the brain activity signals according to the functional near infrared spectroscopy. Not only the functional near infrared spectroscopy but also various methods such as the fMRI method, the EEG method, and the MEG method can be applied to the detection of a brain activity signal.

The control apparatus 3 generates a BCI signal on the basis of the brain activity signal detected by the measuring apparatus 2 and controls a desired control target. The BCI signal is an estimation value of brain activity used in the control. The control apparatus 3 is a computer that controls the measuring apparatus 2 and processes the brain activity signal to control the control target. Therefore, the control apparatus 3 includes a central processing unit (CPU) 8, a main memory 9 configuring a work area of the central processing unit 8, and a storage device unit 10 including a hard disk device. Programs necessary for executing processing by the control system 1, various data necessary for execution of the programs, and the like are stored in the storage device unit 10. The programs are stored in the storage device unit 10 in advance and provided. However, instead, the programs may be recorded in various recording media such as an optical disk and a memory card and provided to the control apparatus 3. Further, the programs may be provided by download through a network such as the Internet.

The control apparatus 3 further includes an input and output interface 11 that inputs and outputs data to and from the measuring apparatus 2, an operation input unit 12 such as a keyboard that inputs operation of the user, an image processing and output unit 14 that drives a display device 13 to display a desired image, a sound processing unit 16 that drives a speaker 15, and an external network interface 17 connected to a network such as the Internet.

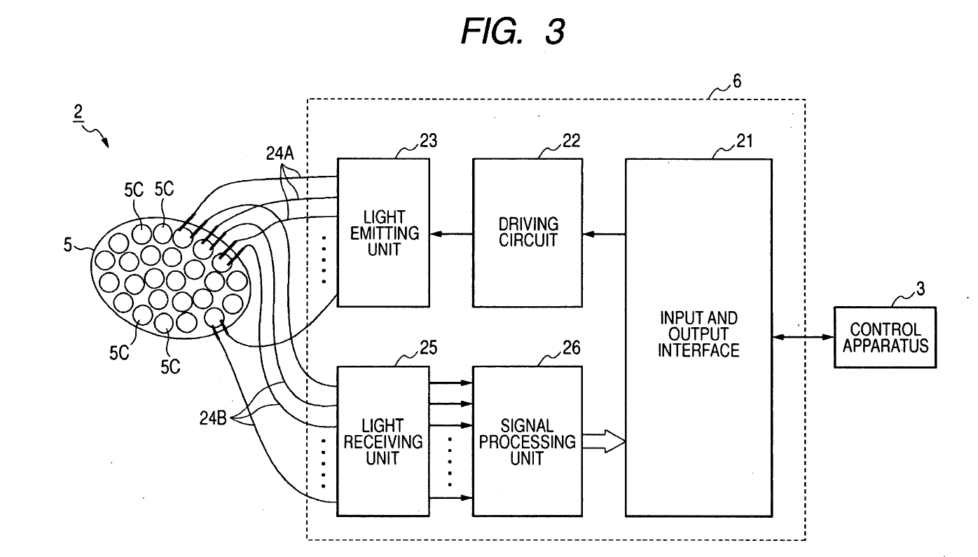

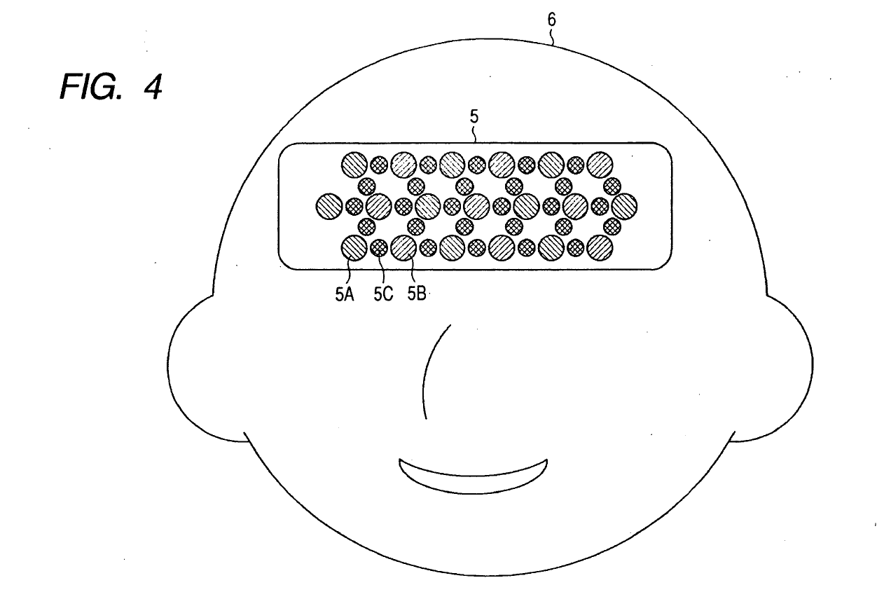

FIG. 3 is a block diagram of details of the measuring apparatus 2. FIG. 4 is a plan view for explaining the measuring device 5. In an example shown in the figure, a brain activity signal from the frontal lobe is detected. In the measuring device 5 (FIG. 4), projecting units 5A that emit near infrared rays to the brain surface layer of the user 4 and light receiving units 5B that receive the near infrared rays emitted to the outside of the head are arranged across measurement regions 5C. In the example shown in FIG. 4, one projecting unit 5A and one light receiving unit 5B are arranged to cover plural measurement regions 5C. For example, brain activity signals in the measurement regions 5C are detected by time-division processing.

In the measuring apparatus 2 (FIG. 3), a measuring apparatus main body unit 6 drives a light emitting unit 23 with a driving circuit 22 according to the control of the control apparatus 3 via the input and output interface 21 and generates a near infrared ray with the light emitting unit 23. The measuring apparatus main body unit 6 leads the near infrared ray generated by the light emitting unit 23 to the measuring device 5 through an optical fiber 24A and emits the near infrared ray from the projecting units 5A. The measuring apparatus main body unit 6 leads the near infrared ray received by the light receiving units 5B to a light receiving unit 25 through an optical fiber 24B and detects a light amount of the near infrared ray received by the light receiving units 5B with the light receiving unit 25. The measuring apparatus main body unit 6 inputs an output signal of the light receiving unit 25, which is a result of the light amount detection, to a signal processing unit 26.

The signal processing unit 26 subjects the output signal of the light receiving unit 25 to analog to digital conversion processing and generates a brain activity signal formed by a digital signal. After removing noise components such as pulse wave components from the brain activity signal, the signal processing unit 26 outputs the brain activity signal to the control apparatus 3 via the input and output interface 21.

The control apparatus 3 processes the brain activity signal output from the measuring apparatus 2 to generate a BCI signal and controls a control target. When the measuring apparatus 2 detects a brain activity signal according to hemoglobin concentration in a blood flow, the control apparatus 3 executes a series of processing explained later directly using the brain activity signal output from the measuring apparatus 2. On the other hand, when the measuring apparatus 2 detects a change amount of hemoglobin concentration as a brain activity signal, the control apparatus 3 executes the series of processing using a change amount of the brain activity signal with respect to a reference value detected in advance in measurement regions. The reference value is a brain activity signal detected in a state in which the user 4 is rested by not giving a stimulus to the user 4. In the following explanation, a configuration of the control apparatus 3 is explained concerning the case in which the measuring apparatus 2 detects a brain activity signal according to hemoglobin concentration in a blood flow. Explanation is omitted concerning the case in which a change amount of hemoglobin concentration is detected as a brain activity signal.

As the hemoglobin concentration, only oxygenated hemoglobin concentration or deoxygenated hemoglobin concentration may be measured. However, in order to more accurately estimate a brain activity state, it is desirable to measure both the oxygenated hemoglobin concentration and the deoxygenated hemoglobin concentration.

The measurement regions 5C form measurement channels for brain activity signals, respectively. In order to accurately estimate a brain activity state, it is necessary to provide about at least ten to twenty measurement channels.

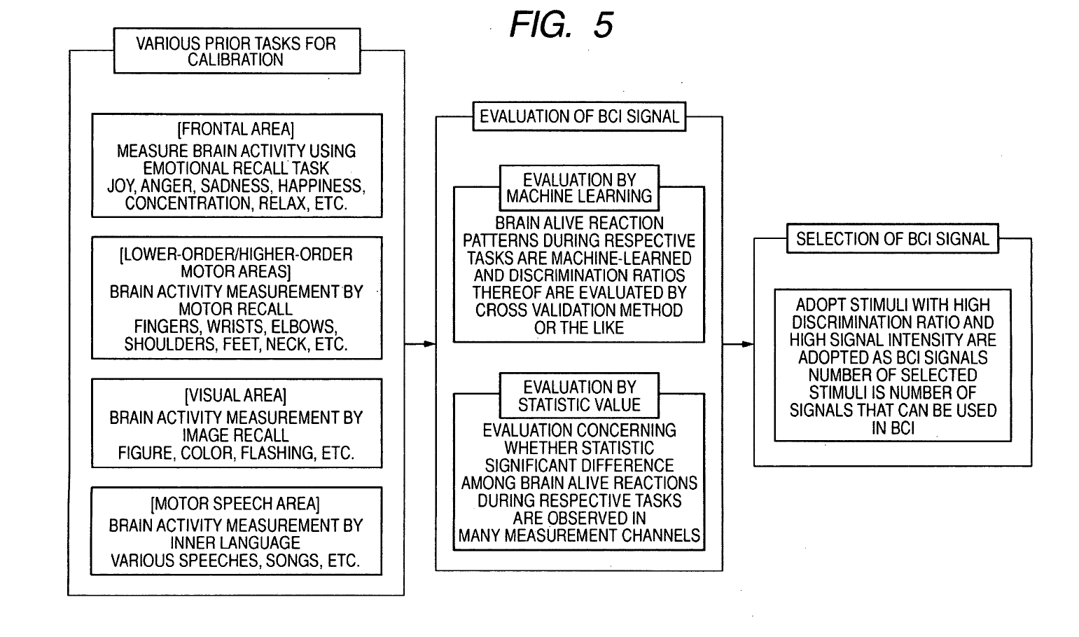

### (2) Prior Processing (Redundant Channel Detection Processing, FIGS. 5 to 7)

Among the measurement channels formed by the measurement regions 5C, redundant measurement channels that may be unable to put a brain activity signal to use in control could be present. Therefore, the central processing unit 8 detects redundant measurement channels for each user 4 according to redundant channel detection processing in prior processing and excludes the redundant measurement channel from a processing target to simplify processing.

In this redundant channel detection processing, as shown in FIG. 5, the central processing unit 8 detects a brain activity signal in a state in which stimuli of various prior tasks corresponding to control are given to the user 4 and detects redundant channels according to processing of the brain activity signal.

In the frontal area, brain activity signals related to human emotions such as joy, anger, sadness, concentration, and relax can be detected by measuring a brain activity signal using an emotion recall task. In the lower-order and higher-order motor areas, brain activity signals related to motions of the regions of the human body such as fingers, wrists, elbows, shoulders, feet, and neck can be detected by measuring a brain activity signal formed by motor recall. In the visual area, brain activity signals in recall of a specific figure, a specific color, flashing, and the like can be detected by measuring a brain activity signal formed by image recall. In the motor speech area, brain activity signals during speech, singing, and the like can be detected by measuring a brain activity signal formed by inner language.

Therefore, in measuring a brain activity signal using the emotion recall task, the central processing unit 8 displays images for causing the user 4 to recall emotions of joy, anger, sadness, concentration, and relax, respectively, on the display device 13 and causes the user 4 to look at the images to thereby give a stimulus to the user 4. In measuring a brain activity signal using the motor recall, the central processing unit 8 causes the user 4 to actually move a region of the human body corresponding thereto or causes the user 4 to image this motion to thereby give a stimulus to the user 4. In measuring the brain activity signal formed by image recall, the central processing unit 8 causes the user 4 to recall an image corresponding thereto to thereby give a stimulus to the user 4. In measuring a brain activity signal formed by inner language, the central processing unit 8 causes the user 4 to, for example, actually speak to thereby give a stimulus to the user 4.

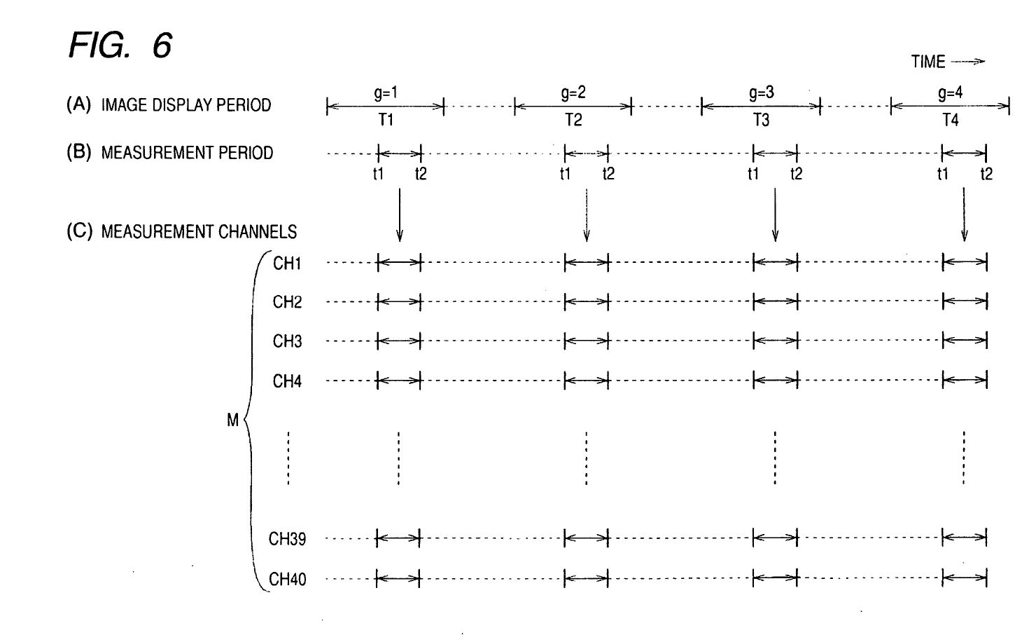

FIG. 6 is a time chart of processing for measuring a brain activity signal when an emotion recall task is used. In an example shown in FIG. 6, the central processing unit 8 displays images (g=1 to 4) corresponding to the respective stimuli on the display device 13 for image display periods indicated by periods T1 to T4 at fixed time intervals to thereby give stimuli of various prior tasks corresponding to control to the user 4. A fixed time lag is present with respect to a stimulus in a brain activity signal of a human. Therefore, after giving the stimuli, the central processing unit 8 acquires brain activity signals detected by the measuring apparatus 2 and records the brain activity signals in the storage device unit 10 in a period from a point t1 when a fixed time elapses to a point t2. In the example shown in FIG. 6, measurement channels are forty channels indicated by CH1 to CH40.

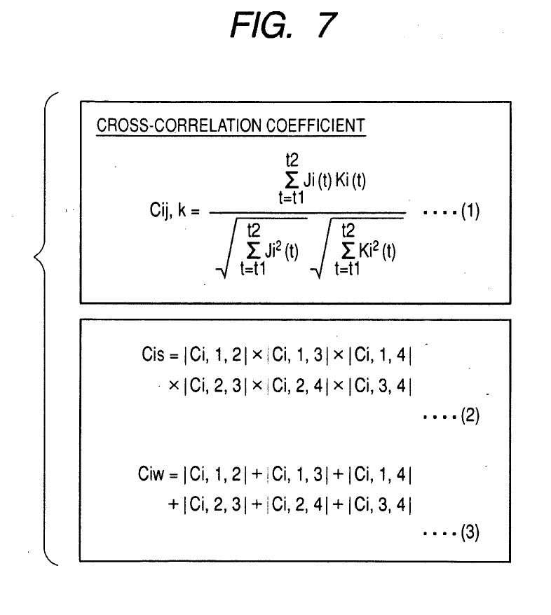

After recording the brain activity signals due to the stimuli in the storage device unit 10, the central processing unit 8 executes arithmetic processing of Formula (1) in FIG. 7 using the recorded brain activity signals and calculates a cross-correlation coefficient Ci, j, k. “i” is a number of a measurement channel and “j” and “k” are numbers of stimuli (j≠K). t1 and t2 are a measurement start point and a measurement end point in FIG. 7. Ji(t) is a signal level (hemoglobin concentration) of a brain activity signal at the point t when the stimulus “j” is given. Ki(t) is a signal level (hemoglobin concentration) of a brain activity signal at the point t when the stimulus “k” is given.

When an absolute value of the cross-correlation coefficient Ci, j, k represented by Formula (1) is close to 1, the measurement channel “i” normally correlates to the stimulus “j” and the stimulus “k”. Therefore, in the measurement channel “i”, the hemoglobin concentration rarely changes when the stimulus “j” is given and when the stimulus “k” is given. It can be said that the measurement channel “i” is a redundant channel concerning identification of the stimuli “j” and “k”.

Consequently, the central processing unit 8 calculates the cross-correlation coefficient Ci, j, k in a combination of all stimuli for each of the measurement channels and evaluates the calculated cross-correlation coefficient Ci, j, k according to a predetermined criteria value. The central processing unit 8 detects, on the basis of a result of this evaluation, a measurement channel having the cross-correlation coefficient Ci, j, k close to a value 1 in the combination of all the stimuli and sets the detected measurement channel as a redundant channel.

A method of determining whether a measurement channel is a redundant channel is not limited to the method explained above. Various methods can be applied as the method. Specifically, as indicated by Formula (2) in FIG. 7, it is also possible that cross-correlation coefficients Ci, j, k detected in the measurement channel “i” are multiplied together and accumulated and an accumulated cross-correlation coefficient Cis is evaluated according to a threshold to detect a redundant channel. Further, as indicated by Formula (3) in FIG. 7, it is also possible that cross-correlation coefficients Ci, j, k detected in the measurement channel “i” are added up and accumulated and an accumulated cross-correlation coefficient Ciw is evaluated according to a threshold to detect a redundant channel.

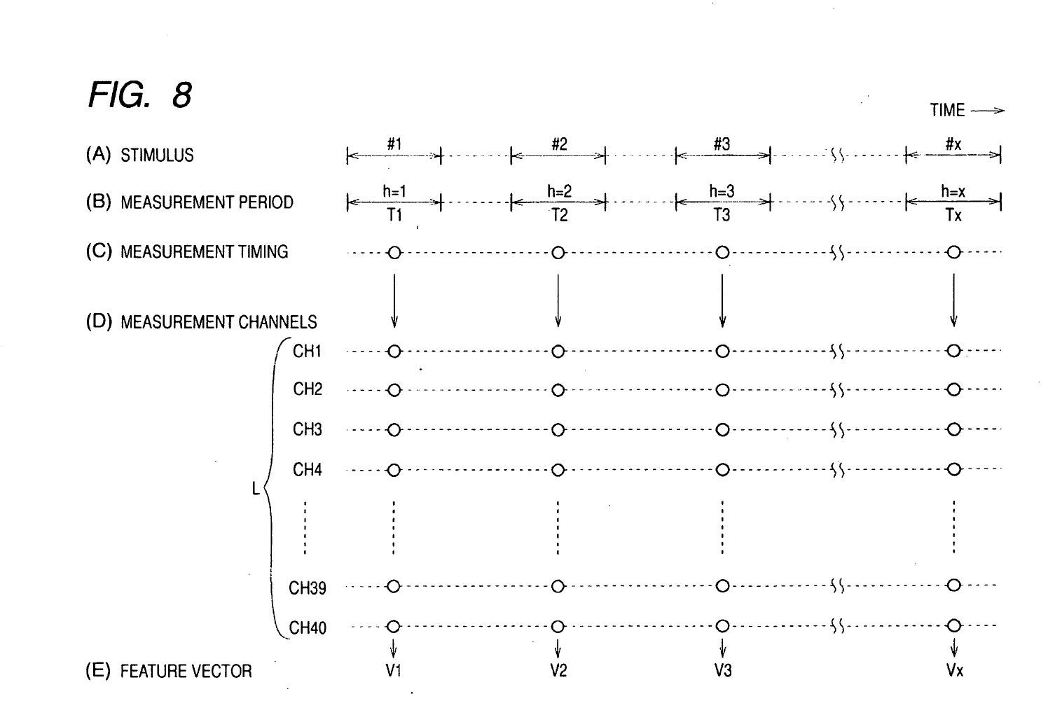

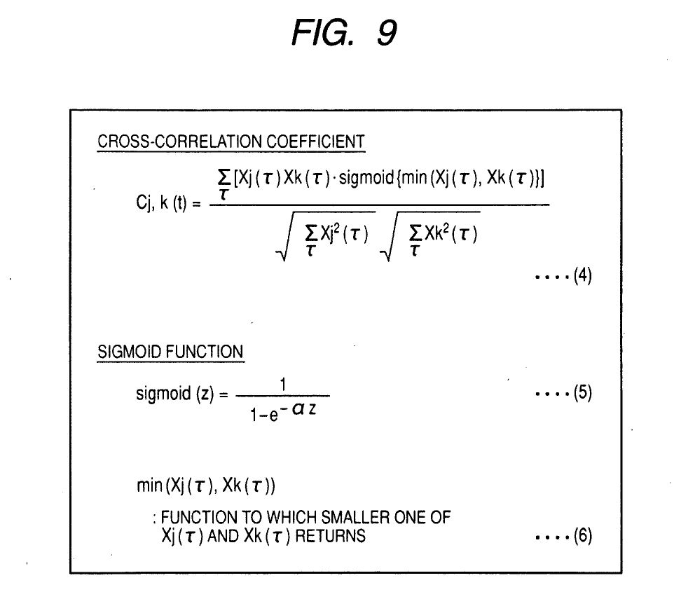

### (3) Prior Processing (Estimation Preparation Processing, FIGS. 8 and 9)

A brain activity signal has an individual difference. Therefore, the control system 1 detects a tendency of the user 4 according to an estimation preparation step in the prior processing. This estimation preparation processing is executed on measurement channels excluding the redundant channel detected in the redundant channel detection processing.

As shown in FIG. 8 in comparison with FIG. 6, the central processing unit 8 detects a brain activity signal using stimuli of various prior tasks corresponding to control and stores the detected brain activity signal in the storage device unit 10. In this case, the brain activity signal used in the redundant channel detection processing may be used.

The central processing unit 8 executes arithmetic processing of Formula (4) in FIG. 9 using the brain activity signal stored in the storage device unit 10 and detects a cross-correlation coefficient Cj, k(t). The cross-correlation coefficient Cj, k(t) indicates a cross-correlation coefficient between the measurement channels “j” and “k” at the point t. In Formula (4), Xj(τ) is a signal level (hemoglobin concentration) of the brain activity signal at a point τ near the point t of the measurement channel “j”. Xk(τ) is a signal level (hemoglobin concentration) of the brain activity signal at a point τ near the point t of the measurement channel “k”. An integral range of Formula (4) is the number of samplings of the brain activity signal at measurement timing specified by the point t. Further, sigmoid is a sigmoid function indicated by Formula (5). In general, the sigmoid function is α=1 in Formula (5). However, in this embodiment, α is set to an appropriate value that is not 1.

The central processing unit 8 calculates feature vectors V1, V2, . . . , and Vx, which characterize a brain alive reaction pattern with respect to a stimulus, using the cross-correlation coefficient Cj, k(t). The feature vectors V are multi-dimensional vectors in which cross-correlation coefficients Cj, k(t) calculated from combinations of the measurement channels, respectively, are elements. The central processing unit 8 detects a feature vector for each stimulus.

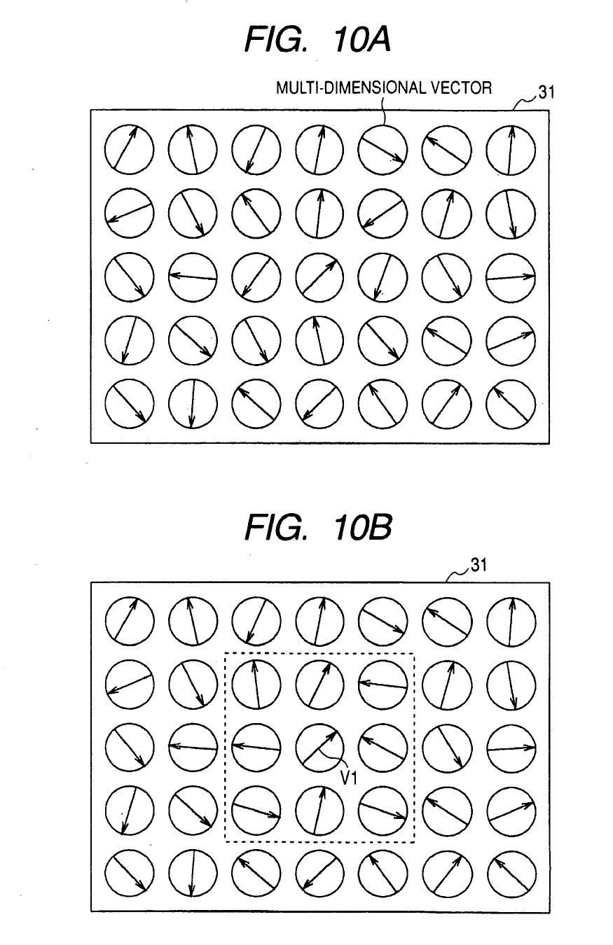

### (4) Prior Processing (Clustering Processing, FIGS. 10A and 10B)

The central processing unit 8 clusters the detected feature vectors V1, V2, . . . , and Vx according to machine learning. The clustering is executed by, for example, self-organizing mapping (SOM). Various methods such as the neural network method, the support vector machine method, the hidden Markov model method, and the boosting method can be applied to the machine learning.

The central processing unit 8 sequentially maps the feature vectors V1, V2, . . . , and Vx to a map in an initial state. The map in the initial state is formed by arranging at random multi-dimensional vector having the same feature vector and the same number of dimensions and having different values. FIG. 10A is a schematic diagram of the map in the initial state. In FIG. 10A, encircled arrows indicate the multi-dimensional vectors, respectively. In FIG. 10A, values of the multi-dimensional vectors are indicated by directions of the arrows.

The central processing unit 8 selects one feature vector V1 out of the feature vectors V1, V2, . . . , and Vx detected in the estimation preparation processing and selects a multi-dimensional vector having a value closest to the selected feature vector V1 from a map 31. The central processing unit 8 maps the feature vector V1 to a coordinate of the selected multi-dimensional vector to thereby replace the selected multi-dimensional vector with the feature vector V1.

FIG. 10B is a diagram of a state in which the feature vector V1 is mapped to a center position of the map 31 in the initial state shown in FIG. 10A. Subsequently, the central processing unit 8 corrects values of multi-dimensional vectors arranged near this mapping position to be close to a value of the feature vector V1. In an example shown in FIG. 10B, the central processing unit 8 corrects values of eight multi-dimensional vectors adjacent to a mapping position surrounded by a broken line.

Subsequently, the central processing unit 8 repeats the same processing for the remaining feature vectors V2, V3, . . . , and Vx and maps all the feature vectors V1 to Vx to the map 31. Consequently, the central processing unit 8 creates clusters corresponding to the feature vectors V1 to Vx on the map 31.

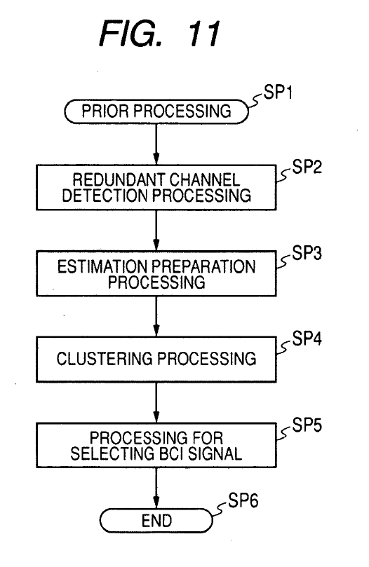

### (5) Prior Processing (Selection of a BCI Signal, FIGS. 5 and 11)

In this embodiment, the central processing unit 8 creates representative vectors that represent characteristics of brain alive reaction patterns with respect to stimuli from multi-dimensional vectors of the respective clusters. The central processing unit 8 compares the representative vectors of the respective clusters and feature vectors calculated from actual stimuli and generates an estimation value for estimating an actual stimulus for each of the stimuli given in the prior processing. Therefore, for example, when three kinds of feature vectors V1 to V3 are detected according to emotional recall tasks of joy, anger, and sadness and the series of prior processing is executed beforehand, in association with this prior stimuli, an estimation value for estimating that an actual stimulus is joy, an estimation value for estimating that an actual stimulus is anger, and an estimation value for estimating that an actual stimulus is sadness are detected for each of actual stimuli. As a method of calculating an estimation value, it is possible to apply various methods such as a method of calculating an estimation value by normalizing a distance between a representative vector and a feature vector with the length of the representative vector and a method of calculating a probability that feature vectors are included in the respective clusters.

The central processing unit 8 controls a desired control target on the basis of this estimation value. As the representative vectors, the feature vectors of the respective clusters can be applied. However, the same stimulus may be repeated to detect plural feature vectors by the same stimulus and map the plural feature vectors to create a cluster. In this case, a feature vector in a position closest to the center of gravity of the cluster or a feature vector most characteristic in the cluster is extracted as a representative vector.

The estimation values of the respective stimuli generated in this way are highly independent and can be used for control. However, all the estimation values do not always have high independency. Therefore, as shown in FIG. 5, the central processing unit 8 evaluates a determination ratio by these estimation values and selects a highly independent estimation value in determination processing for a BCI signal. More specifically, the central processing unit 8 detects, according to the cross validation method performed by using the representative vectors of the respective clusters, representative vectors from which significant estimation values can be detected. Further, the central processing unit 8 selects a representative vector from the detected representative vectors using a statistic value and sets an estimation value calculated from this representative vector in the BCI signal. Processing of the selection by the statistic value is executed between brain alive reactions during respective tasks by selecting a representative vector, a statistic significance difference of which is detected in many measurement channels.

When the same stimulus is repeated and plural feature vectors by the same stimulus are mapped and clustered, it is also possible that feature vectors of the respective clusters are set as representative vectors, respectively, the respective clusters are represented by a representative vector group, and an estimation value is generated according to comparison of the representative vector group and feature vectors by actual stimuli.

In the selection of the BCI signal, it is desirable to measure a brain alive reaction during task recall that is trained to a certain degree by using plural tasks by right-hand grasping recall, emotions recall, and the like.

FIG. 11 is a flowchart of a prior processing procedure of the central processing unit 8. In the prior processing, the central processing unit 8 detects a redundant channel according to redundant channel detection processing in step SP2 and excludes the redundant channel from a processing target. The central processing unit 8 detects a feature vector for each of stimuli according to estimation preparation processing in the following step SP3 and maps the detected feature vector to a map according to clustering processing in the following step SP4. Further, the central processing unit 8 sets a BCI signal according to BCI signal selection processing in the following step SP5 and then finishes the prior processing.

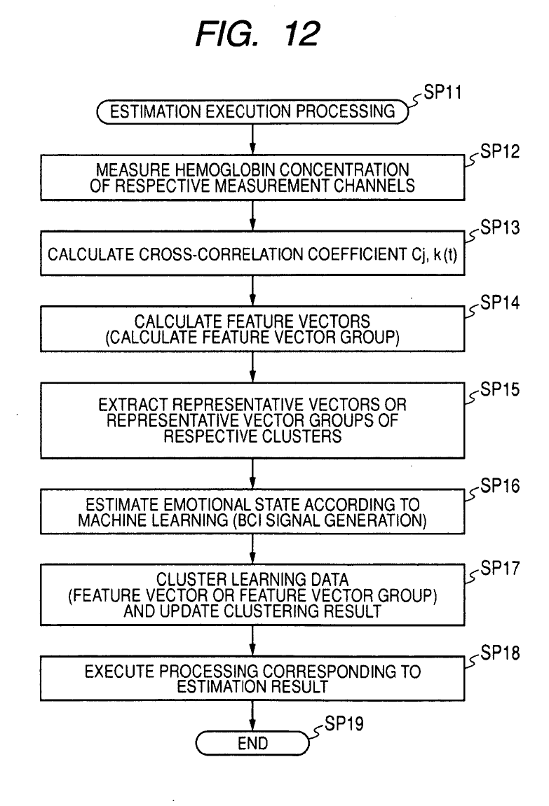

### (6) Estimation Execution Processing (FIG. 12)

FIG. 12 is a flowchart of a processing procedure of estimation execution processing for processing actual stimuli. After starting control of a control target, the central processing unit 8 executes this processing procedure at fixed time intervals. After starting this processing procedure, the central processing unit 8 shifts from step SP11 to step SP12 and measures a brain activity signal by controlling the measuring apparatus 2. Subsequently, in the following step SP13, the central processing unit 8 executes arithmetic processing of Formula (4) using the brain activity signal excluding a redundant channel and calculates the cross-correlation coefficient Cj, k(t).

In the subsequent step SP14, the central processing unit 8 detects a feature vector using the calculated cross-correlation coefficient Cj, k(t). The central processing unit 8 may detect plural feature vectors by repeating the detection of a brain activity signal and detect a feature vector group.

Subsequently, the central processing unit 8 shifts to step SP15 and detects representative vectors or a representative vector group of the respective clusters formed on the map. In the following step SP16, the central processing unit 8 compares the feature vector and the representative vectors according to machine learning of a back propagation neural network or the like in which the representative vectors and the feature vector are used as teacher data and learning data and detects an estimation value of an actual stimulus for each of stimuli of the respective clusters formed on the map. In this processing, the central processing unit 8 generates estimation values of actual stimuli only for clusters related to the estimation value set in the BCI signal to thereby generate a BCI signal.

Subsequently, the central processing unit 8 shifts to step SP17, maps the feature vector as the learning data onto the map 31 according to the self-organizing mapping, and updates a clustering result. Consequently, the central processing unit 8 improves accuracy in the following estimation execution processing. In the subsequent step SP18, the central processing unit 8 executes processing corresponding to the BCI signal detected in step SP17. Thereafter, the central processing unit 8 shifts to step SP19 and finishes the processing procedure.

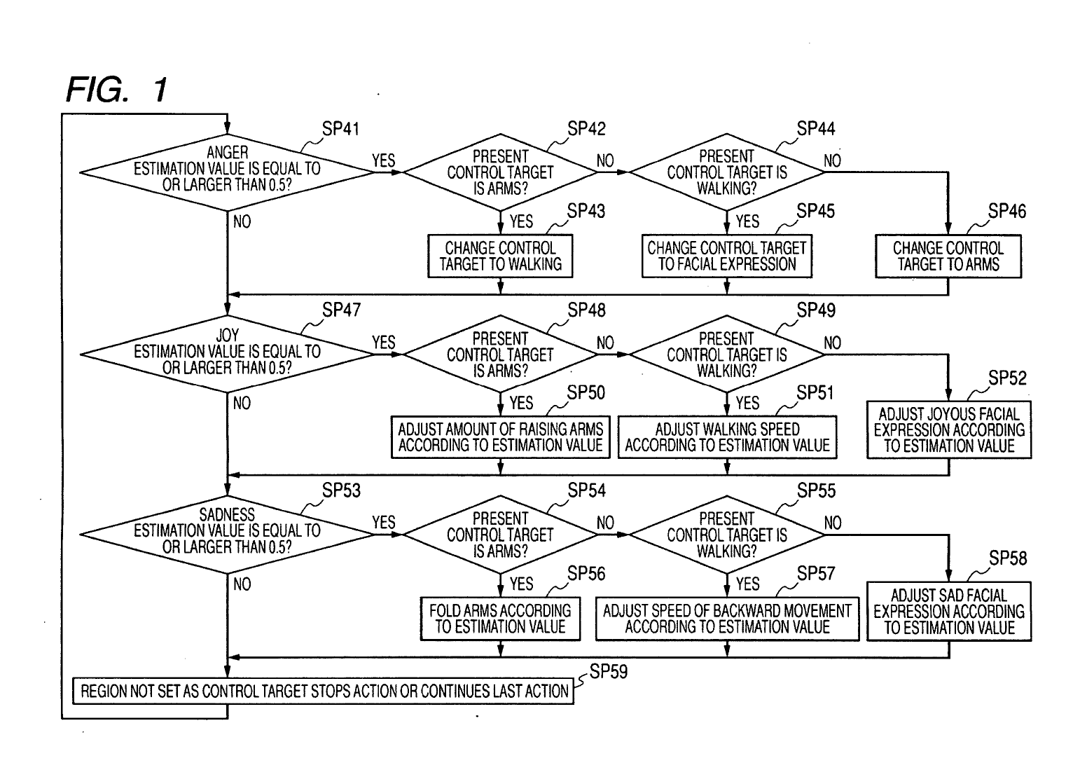

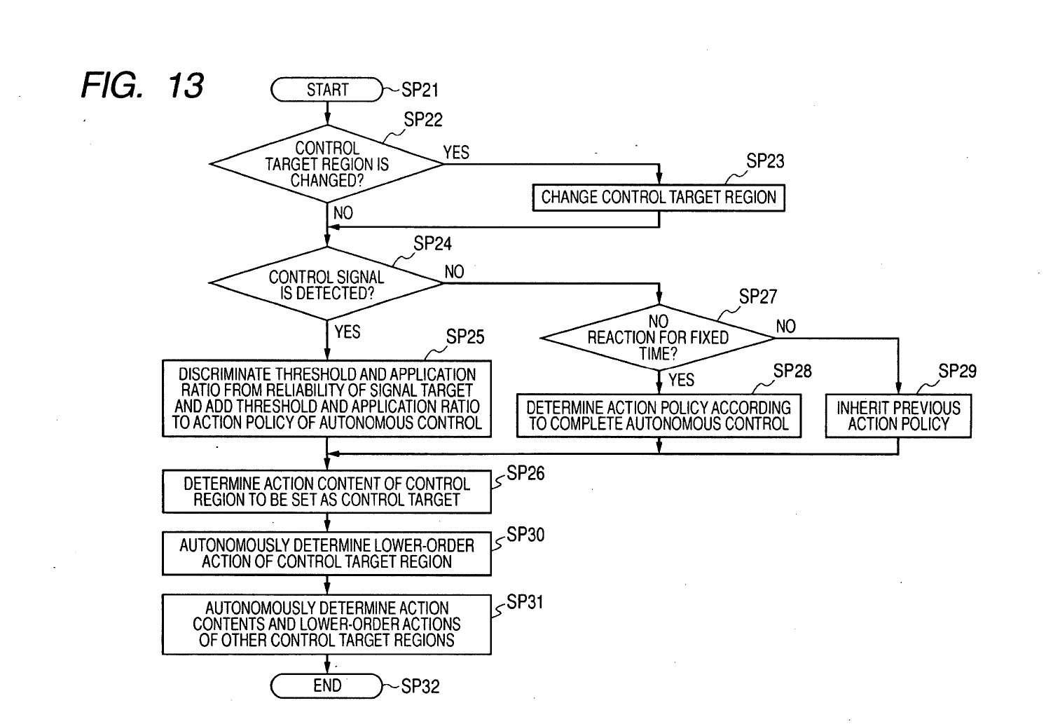

### (7) Control of a Single Agent (FIGS. 1 and 13)

In this embodiment, a human-type agent classified into a so-called single agent is set as a control target of the control apparatus 3. The central processing unit 8 builds up this human-type agent and displays the human-type agent on the display device 13.

According to the series of processing explained above, it is possible to estimate a present multi-dimensional thinking state of the user. However, when a BCI signal is applied to control of a single agent having a large number of degrees of freedom, inmost cases, a degree of freedom of an actual control target is large compared with a degree of freedom expected with the BCI signal. The number of degrees of freedom is the number of outputs that can be represented by an agent such as a degree of freedom of a body or the number of sounds that can be uttered.

More specifically, for example, when walk control in a general human-type agent is assumed, if respective arms have a degree of freedom of 6 and legs have a degree of freedom of 6, the human-type agent has a degree of freedom as high as 24 as a whole. Therefore, it can be said that it is nearly difficult to control all the degrees of freedom with the BCI signal.

Therefore, in this embodiment, first, a BCI signal is generated for only higher-order action control. Consequently, only the higher-order action control is performed by the BCI signal. Second, after a degree of freedom of a control target is selectively set, lower-order action control is executed only for a specific region by the BCI signal. Consequently, in this embodiment, a single agent having a large number of degrees of freedom compared with a degree of freedom expected with the BCI signal is controlled. When one action control includes the other action control, one action control is defined as higher-order action control and the other is defined as lower-order action control. Control content of the human-type agent in this embodiment is formed in a hierarchical structure from primitive lower-hierarchy control to higher-order control such as an action policy.

More specifically, in the prior processing, a BCI signal is set according to a prior task corresponding to highest-order action control. When, for example, three BCI signals are set according to the prior task, a control target region is switched by using a specific BCI signal among the three BCI signals and the switched control target region is controlled by all the BCI signals or the remaining BCI signals.

It goes without saying that the three BCI signals are used for higher-order action control performed by each of the BCI signals. It goes without saying that, in the control of the control target region by the remaining BCI signals, control of a control target region related to lower-order action control is control corresponding to high-order action control by a BCI signal corresponding to the control. Therefore, for example, when three BCI signals are generated by three facial expressions of a joyous face, a sad face, and an expressionless face, action control for walking as action control lower in order than the facial expressions represents emotions of joy, sadness, and emotionlessness corresponding to the joyous face, the sad face, and the expressionless face, respectively.

In this embodiment, the human-type agent as the control target has three control target regions, i.e., arms, walking, and facial expression and can perform, concerning the arms, three kinds of control for raising the arms, lowering the arms, and folding arms. Concerning the walking, the human-type agent can perform three kinds of control for moving forward, moving backward, and stopping. Concerning the facial expression, the human-type agent can perform three kinds of control for the joyous face, the sad face, and the expressionless face. As a whole, the number of degrees of freedom is set to nine. In the human-type agent, selection concerning whether a control target region is the face, the arms, or the walking is highest-order control. Control of specific facial expression and the like in the respective control target regions is lower-order control.

Therefore, in the prior processing, the central processing unit 8 sets estimation values corresponding to emotional recall tasks of joy, anger, and sadness in BCI signals. In this case, the central processing unit 8 cyclically switches the control target regions sequentially in order of the arms, the walking, and the facial expression according to, for example, the BCI signal of anger. The central processing unit 8 controls the switched control target region according to the BCI signal of joy or sadness. Consequently, the central processing unit 8 controls a single agent having a large number of degrees of freedom compared with the number of BCI signals.

FIG. 13 is a flowchart of a control procedure of the central processing unit 8 performed by using the BCI signals. The processing in step SP18 in FIG. 12 is shown in detail in FIG. 13. After starting this processing procedure, the central processing unit 8 shifts from step SP21 to step SP22 and determines, for example, whether the control target region should be changed according to the BCI signal of anger. The determination is executed by, for example, generating an estimation value forming the BCI signal in a range of values 1 to 0 and evaluating this estimation value according to a specific threshold.

When an affirmative result is obtained, the central processing unit 8 shifts from step SP22 to step SP23 and, after switching the control target region, shifts to step SP24. When a negative result is obtained in step SP22, the central processing unit 8 directly shifts from step SP22 to step SP24.

In step SP24, the central processing unit 8 determines, for example, whether the control target region should be changed according to the remaining BCI signals in the same manner as step SP22. When it is determined that a change of a control amount is necessary, the central processing unit 8 shifts from step SP24 to step SP25 and sets, according to the BCI signal used in the determination in step SP24, a control amount for a control target region selected at that point. In this case, for example, if control for raising and lowering the arms is performed, the center of gravity may change to make walking difficult. Therefore, it is desirable that the single agent to be controlled can perform autonomous control to a certain extent according to the control amount in step SP25.

After completing the processing in step SP25, the central processing unit 8 shifts to step SP26. On the other hand, when it is determined in step SP24 that the change of the control amount is unnecessary, the central processing unit 8 shifts from step SP24 to step SP27. The central processing unit 8 determines, concerning the remaining BCI signals, whether there is no change for a fixed time or longer.

When an affirmative result is obtained, the central processing unit 8 shifts from step SP27 to step SP28, sets a control amount according to complete autonomous control, and shifts to step SP26. When a negative result is obtained in step SP27, the central processing unit 8 shifts from step SP27 to step SP29 and, after setting the actions performed to that point to be continued, shifts to step SP26.

In the control system 1, the BCI signals may be interrupted by a fall in power of concentration of the user 4 during BCI control, mixing of external noise, a trouble of the measuring apparatus 2, or the like. In such a case, the central processing unit 8 obtains the affirmative result in step SP27. Therefore, in this embodiment, when a significant signal is not detected for a fixed time, the central processing unit 8 switches the control target to an autonomous control mode according to the processing in steps SP27 and SP28. When a significant signal is obtained, the central processing unit 8 switches the control target to the BCI control mode again. In this way, the central processing unit 8 flexibly copes with the fall in power of concentration and the like.

As action determination in the autonomous control mode, it is conceivable that the central processing unit 8 maintains the last control, conforms to a policy of action determination set by the user beforehand, causes the control target to perform an action conforming to control in a situation closest from a control history to that point, or does not cause the control target to action. When the central processing unit 8 maintains the last control, this is effective when the BCI signals are interrupted. When the central processing unit 8 conforms to the policy of action determination set by the user beforehand, it is necessary to set the action control beforehand. When the central processing unit 8 indirectly controls the control target via an external information apparatus, the control may be left to an algorithm of the external information apparatus. When the central processing unit 8 indirectly controls the control target via the external information apparatus, for example, the control target is a robot that can actually move or is operation of a game or the like.

After determining action content related to the control target region in step SP26, in the subsequent step SP30, the central processing unit 8 autonomously set a lower-order action as specific control of the control garget region. In the subsequent step SP31, the central processing unit 8 autonomously sets lower-order actions of other control target regions to correspond to the setting in step SP30. The central processing unit 8 shifts to step SP32 and finishes the processing procedure.

FIG. 1 is a flowchart of control by the central processing unit 8 according to repetition of the processing in FIG. 13. FIG. 1 is a diagram of a relation between regions as control targets and the control.

In step SP41 in FIG. 1, the central processing unit 8 determines whether a BCI signal formed by an estimation value of anger is equal to or larger than a value 0.5 to thereby determine, as explained in step SP22, whether a control target region should be changed.

When an affirmative result is obtained, the central processing unit 8 shifts to step SP42 and determines whether the present control target region is the arms. When an affirmative result is obtained in step SP42, the central processing unit 8 shifts to step SP43 and changes the control target region to the walking. When a negative result is obtained in step SP42, the central processing unit 8 shifts to step SP44 and determines whether the present control target region is the walking. When an affirmative result is obtained in step SP44, the central processing unit 8 shifts from step SP44 to step SP45 and switches the control target region to the facial expression. When a negative result is obtained in step SP44, the central processing unit 8 shifts from step SP44 to step SP46 and switches the control target region to the arms. Consequently, the central processing unit 8 cyclically switches the control target region sequentially according to the BCI signal of anger as explained above concerning step SP23.

When a negative result is obtained in step SP41, the central processing unit 8 shifts to step SP47. When the processing in step SP43, the processing in step SP45, or the processing in step SP46 is finished, the central processing unit 8 shifts to step SP47. In step SP47, the central processing unit 8 determines whether a BCI signal formed by an estimation value of joy is equal to or larger than a value 0.5. Consequently, the central processing unit 8 determines whether the control target region should be changed according to the remaining BCI signal as explained above concerning step SP24.

When an affirmative result is obtained in step SP47, the central processing unit 8 shifts to step SP48 and determines whether the present control target region is the arms. When a negative result is obtained in step SP48, the central processing unit 8 shifts from step SP48 to step SP49 and determines whether the present control target region is the walking. When an affirmative result is obtained in step SP48, the central processing unit 8 shifts from step SP48 to step SP50 and sets a control amount for raising the arms according to an estimation value of joy. When an affirmative result is obtained in step SP49, the central processing unit 8 shifts from step SP49 to step SP51 and sets a control amount for walking speed of forward movement according to an estimation value of joy. When a negative result is obtained in step SP49, the central processing unit 8 shifts from step SP49 to step SP52 and sets the facial expression in an expression of joy according to the estimation value of joy. Consequently, the central processing unit 8 executes the processing in step SP25 on the BCI signal of joy according to the processing in steps SP48 to SP52 and controls, according to the BCI signal of joy, the control target region selected by the BCI signal of anger.

When a negative result is obtained in step SP47, the central processing unit 8 shifts to step SP53. When the processing in step SP50, the processing in step SP51, or the processing in step SP52 is finished, the central processing unit 8 shifts to step SP53. In step SP53, the central processing unit 8 determines whether a BCI signal formed by an estimation value of sadness as the remaining BCI signal is equal to or larger than a value 0.5. Consequently, the central processing unit 8 determines whether the control target region should be changed according to the remaining BCI signal as explained above concerning step SP24.

When an affirmative result is obtained in step SP53, the central processing unit 8 shifts to step SP54 and determines whether the present control target region is the arms. When a negative result is obtained in step SP54, the central processing unit 8 shifts from step SP54 to step SP55 and determines whether the present control target region is the walking. When an affirmative result is obtained in step SP54, the central processing unit 8 shifts from step SP54 to step SP56 and sets control to fold the arms according to an estimation value of sadness. When an affirmative result is obtained in step SP55, the central processing unit 8 shifts from step SP55 to step SP57 and sets a control amount for walking speed of backward movement according to the estimation of sadness. When a negative result is obtained in step SP55, the central processing unit 8 shifts from step SP55 to step SP58 and sets the facial expression in an expression of sadness according to the estimation value of sadness. Consequently, the central processing unit 8 executes the processing in step SP25 for the BCI signal of joy according to the processing in steps SP48 to SP52. The central processing unit 8 executes the processing in step SP25 for the BCI signal of sadness according to the processing in steps SP54 to SP58 and controls, according to the BCI signal of sadness, the control target region selected by the BCI signal of anger.

When a negative result is obtained in step SP53, the central processing unit 8 shifts to step SP59. When the processing in step SP56, the processing in step SP57, or the processing in step SP58 is finished, the central processing unit 8 shifts to step SP59. In step SP59, the central processing unit 8 performs setting, concerning a region for which a control amount is not set, to continue the control performed to that point or perform control by autonomous control. Thereafter, the central processing unit 8 returns to step SP41. In this case, the central processing unit 8 may control the control target to stop an action according to necessity.

The facial expression control for expressionlessness is executed by the processing in step SP59 when the processing in step SP52 or SP58 is not executed for a fixed time. The action control for lowering the arms is executed by the processing in step SP59 when the processing in steps SP50 and SP51 is not executed for the fixed time. The action control for stopping the walking is executed by the processing in step SP59 when the processing in steps SP51 and SP52 is not executed for the fixed time. The processing in steps SP28 and SP29 in FIG. 13 includes the processing in step SP59.

In the example explained above, the control target region is switched according to the estimation value of anger and controlled according to the estimation values of joy and sadness. However, the control target region may be switched according to the estimation value of anger and controlled according to the estimation values of joy and sadness.

### (8) Operations According to the Embodiment

With the configuration explained above, in the control system 1 (FIGS. 2 to 4), the measuring apparatus 2 measures a brain activity signal according to the functional near infrared spectroscopy and the central processing unit 8 of the control apparatus 3 processes the brain activity signal and controls the human-type agent. In this control, a measurement channel redundant for identification of brain activity is detected and excluded from a processing target according to prior processing (FIGS. 6, 7, and 11), whereby processing is reduced. A brain activity signal is measured by using a prior task and feature vectors formed by a result of the measurement are mapped to the self-organizing map to detect a tendency of the user 4 (FIGS. 8 to 11).

In the control system 1, brain activity signals by actual stimuli are processed to detect feature vectors. The feature vectors by the actual stimuli and representative vectors of respective stimuli according to the self-organizing map are compared to generate estimation values of the actual stimuli for each of stimuli learned in advance, whereby the actual stimuli are estimated (FIG. 12).

The estimation values of the actual stimuli are highly independent and can be used for control. However, all the estimation values do not always have high independency. Therefore, in the control system 1, in the prior processing, an estimation value having a high discrimination ratio and high signal intensity is further selected from the estimation values of the actual stimuli detected by using the self-organizing map according to determination by machine learning and determination by statistic values. The selected estimation value is set in a BCI signal (FIGS. 5 and 11). The human-type agent as the control target is controlled by using the BCI signal. Consequently, in the control system 1, wrong control can be reduced. The user 4 can operate the control target as intended.

However, in most cases, the BCI signal detected in this way has a small degree of freedom compared with the actual control target. Therefore, it is difficult to immediately apply the BCI signal to control of a control target having a high degree of freedom. Therefore, in the control system 1, a BCI signal is generated only for higher-order action control. The higher-order action control is executed by the BCI signal. Further, after a degree of freedom of a control target is selectively set, action control is executed only for a specific region according to the BCI signal. Consequently, in this embodiment, it is possible to control a single agent having a large number of degrees of freedom compared with a degree of freedom expected with the BCI signal.

More specifically, in the control system 1, BCI signals are set according to a prior task corresponding to the higher-order action control. Control target regions are sequentially switched by a specific BCI signal among the BCI signals and the respective control target regions are controlled by the remaining BCI signals. More specifically, a BCI signal is selected according to emotional recall tasks of anger, joy, and sadness. The control target regions are sequentially switched by a BCI signal of anger among the BCI signals. Consequently, as a whole, the nine degrees of freedom are selectively limited to three degrees of freedom. Action control related to the three degrees of freedom is executed by the remaining BCI signals of joy and sadness.

When control target regions are set in the arms, the walking, and the facial expression, if a stimulus of joy is detected by the BCI signal of joy, action control is executed to raise the arms, increase the walking speed, and change the facial expression to a smiling face. When the control target regions are set in the arms, the walking, and the facial expression, if a stimulus of sadness is detected by the BCI signal of sadness, action control is executed to fold the arms, move backward, and change the facial expression to a sad face When it is difficult to detect both the stimuli of joy and sadness according to the BCI signals of joy and sadness, action control is performed to lower the raised arms, stop the walking, and change the facial expression to expressionlessness.

Consequently, in the control system 1, since the control target regions are cyclically switched sequentially, for example, when a stimulus of joy is detected, it is possible to control the human-type agent to raise the arms and walk at high speed with a smiling face. When a stimulus of sadness is detected, it is possible to control the human-type agent to fold the arms and move backward with a sad face. When a stimulus of anger is detected, it is possible to control the human-type agent to lower the arms and stand expressionlessly.

Further, in the control system 1, when a significant BCI signal is not obtained for the fixed time or longer, the control target is switched to the autonomous control mode. When a significant signal is obtained, the control target is switched to the BCI control mode again. Consequently, in the control system 1, when the BCI signals are interrupted by a fall in power of concentration of the user 4, mixing of external noise, a trouble of the measuring apparatus 2, or the like, it is possible to flexibly cope with the interruption of the BCI signals.

### (9) Effects of the Embodiment

With the configuration explained above, the control target regions are sequentially switched on the basis of estimation values of brain activity related to higher-order action control to execute action control according to the higher-order estimation values. Therefore, it is possible to efficiently use the estimation values of brain activity and surely control a control target having a high degree of freedom.

Since the control target is a single agent, concerning control of the single agent, it is possible to efficiently use the estimation values of brain activity and surely control a control target having a high degree of freedom.

When a significant BCI signal is not obtained for the fixed time or longer, the control target is switched to the autonomous control mode. Therefore, when the BCI signals are interrupted by a fall in power of concentration of the user 4, mixing of external noise, a trouble of the measuring apparatus 2, or the like, it is possible to flexibly cope with the interruption of the BCI signals.

### Second Embodiment

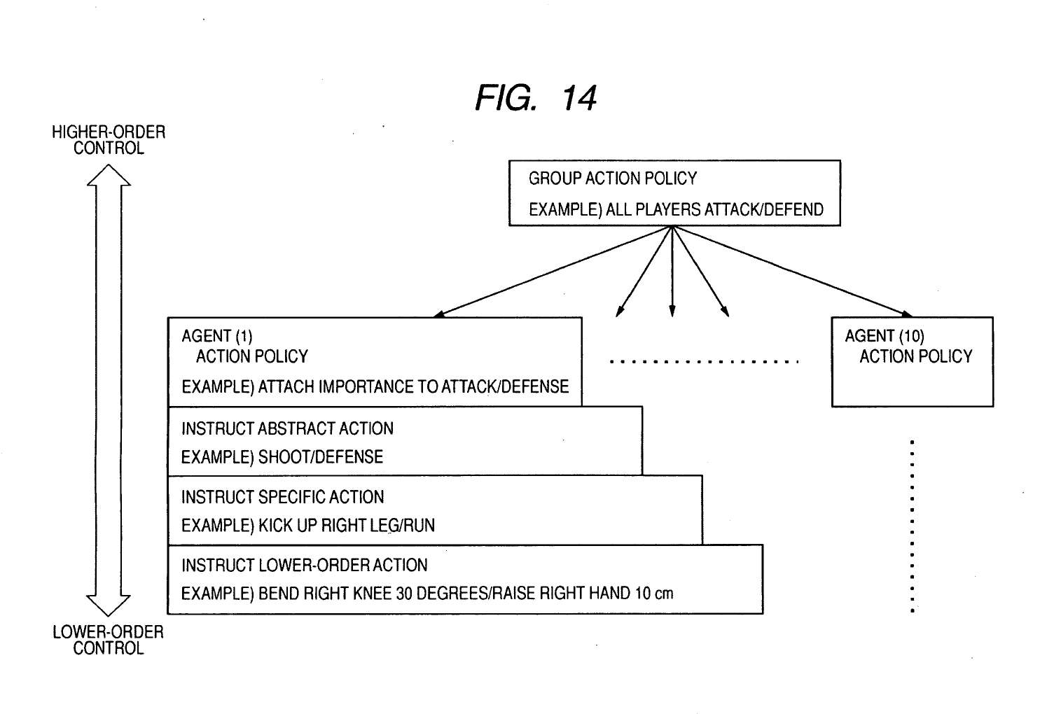

FIG. 14 is a diagram for explaining a control system according to a second embodiment of the present invention. In this embodiment, a multi-agent having plural agents that act autonomously from one another is set as a control target. More specifically, a soccer game played by operating plural players is set as a control target. The control system according to this embodiment is configured the same as the control system according to the first embodiment except that a configuration concerning this control target is different. Therefore, in the following explanation, this embodiment is explained with reference to the configuration shown in FIGS. 2 and 3 as appropriate. Further, the respective agents configuring the multi-agent are referred to as single agents.

In the multi-agent as the control target in this embodiment (FIG. 1), as in the first embodiment, control content of the multi-agent is formed in a hierarchical structure from primitive lower-hierarchy control to higher-order control content such as an action policy. The multi-agent is configured to be autonomously controllable according to the control content in the respective hierarchies. The multi-agent is an agent having a large number of degrees of freedom compared with the number of degrees of freedom that can be treated by a BCI signal.

In this embodiment, as in the first embodiment, a BCI signal is generated only for higher-order action control. Consequently, only the higher-order action control is performed by the BCI signal. Second, after a degree of freedom of a control target is selectively set, lower-order action control is executed only for a specific region according to the BCI signal. Consequently, in this embodiment, concerning control of the multi-agent, single agents having a large number of degrees of freedom compared with a degree of freedom expected with the BCI signal are controlled.

More specifically, in this embodiment, BCI signals are set according to a prior task corresponding to the higher-order action control. Control target regions are switched by using a part of the BCI signals set according to the prior task and the switched control target regions are controlled by all the BCI signals or the remaining BCI signals.

As shown in FIG. 14, the multi-agent in this embodiment is configured to be capable of controlling an overall action according to control by a group action policy and is configured to be capable of controlling offense and defense to instruct all the players to attack or defend. This control is on the premise that the respective agents have an autonomous action determination process for determining an action in each situation of each of the agents, for example, if an agent holds a ball, the agent shoots, and if an opponent holds a ball, an agent takes the ball away from the opponent. Therefore, for example, like a coach, the entire action control gives an abstract instruction to all the agents using a BCI signal and changes the situation.

The control system 1 executes control for attack by all the players according to a BCI signal in a positive thinking state and executes control for defense by all the players according to a BCI signal in a negative thinking state.

In the multi-agent, action control by an action policy of the respective agents for controlling offense and defense of the agents is provided in action control in a lower-order hierarchy of action control by this overall action policy. In this case, action control for a single agent group such as a defense group or an offense group may be provided.

Further, in the multi-agent, action control for abstractly instructing actions of the respective agents such as shoot, defense, and the like is provided in action control in a lower-order hierarchy of the action control by the action policy of the respective agents. Moreover, action control for specifically instructing actions of the respective agents such as kicking up the right leg, running, and the like is provided in a lower-order hierarchy of this action control.

Lower-order action control for instructing actions such as bending the right knee by 30 degrees, raising the right hand 10 cm, and the like is provided in a lowest-order hierarchy.

Concerning these kinds of action control for the respective agents, it is possible to determine a group or an individual to be set as a control target from a motor recall processing result of the legs from the motor area and control actions such as attacking or moving back ward and defending for each of the single agents or each of single agent groups. It is also possible to select shoot, pass, and the like depending on the number of signals that can be controlled by a BCI signal.

Consequently, in the control system 1 according to this embodiment, a BCI signal is se: from the prior process for at least the action control by the overall action policy. However, an estimation value related to action control in a lower-order hierarchy may be further set in the BCI signal. In the following explanation, a BCI signal is generated for action control by a highest-order overall action policy, action control related to action policy of the respective agents in a lower-order hierarchy of the action control related to the overall action policy is executed, and, action control for a lower-order hierarchy is left to the autonomous action control of the multi-agent.

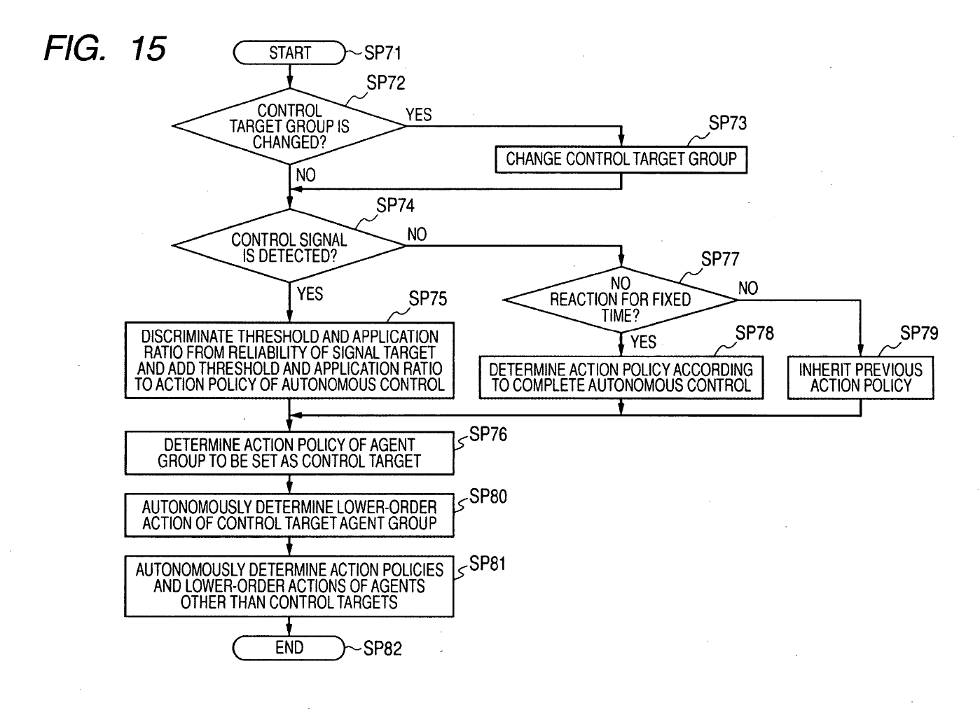

FIG. 15 is a flowchart for explaining a processing procedure of the central processing unit 8 according to the control of the multi-agent in comparison with FIG. 13. The central processing unit 8 executes this processing procedure instead of the processing procedure shown in FIG. 13 to control the multi-agent. After starting this processing procedure, the central processing unit 8 shifts from step SP71 to step SP72. The central processing unit 8 evaluates, for example, a BCI signal in a positive thinking state according to a predetermined threshold and determines whether a control target region should be changed. When an affirmative result is obtained in step SP72, the central processing unit 8 shifts from step SP72 to step SP73 and, after switching the control target region, shifts to step SP74. When a negative result is obtained in step SP72, the central processing unit 8 directly shifts from step SP72 to step SP74. The switching of the control target region is sequential cyclical switching of the action control by the highest-order overall action policy and the action control by the action policy of the respective agents.

In step SP74, the central processing unit 8 evaluates any one of remaining BCI signals and all BCI signals or both, for example, in the same manner as step SP72. When it is determined in SP74 that a control amount needs to be changed, the central processing unit 8 shifts from step SP74 to step SP75 and sets a control amount for a control target region selected at that point according to the BCI signal used in the evaluation in step SP74.

After completing the processing in step SP75, the central processing unit 8 shifts to step SP76. On the other hand, when it is determined in step SP74 that the control amount does not need to be changed, the central processing unit 8 shifts from step SP74 to step SP77. The central processing unit 8 determines whether any one of the remaining BCI signals and all the BCI signals or both do not change for a fixed time.

When an affirmative result is obtained in step SP77, the central processing unit 8 shifts from step SP77 to step SP78, sets a control amount according to complete autonomous control, and shifts to step SP76. When a negative result is obtained in step SP77, the central processing unit 8 shifts from step SP77 to step SP79 and, after setting the actions performed to that point to be continued, shifts to step SP76. Consequently, the central processing unit 8 switches an action mode to the autonomous control mode as appropriate and flexibly copes with a fall in power of concentration and the like.

The central processing unit 8 determines action content related to the control target region in step SP76. In the subsequent step SP80, the central processing unit 8 autonomously sets a lower-order action as specific control of the control target region. In the subsequent step SP81, the central processing unit 8 autonomously sets lower-order actions in other control target regions to correspond to the setting in step SP80, shifts to step SP82, and finishes the processing procedure.

According to this embodiment, concerning the control of the multi-agent, it is possible to obtain effects same as those in the first embodiment.

### Third Embodiment

In the second embodiment, overall and fixed units are controlled according to the estimation value related to the overall action policy. However, the present invention is not limited to this. BCI signals may be allocated to selected individuals, respectively. Specifically, for example, an estimation value of a motion of the motor area or an emotion estimation value detected by the left brain is allocated to a first agent and an estimation value detected by the right brain in the same manner is allocated to a second agent. In the allocation of the estimation values to the agents, the estimation values may be allocated according to various criteria, for example, the estimation value by the left brain is allocated to an agent closest to a ball with respect to a distance from the ball and the estimation value by the right brain is allocated to an agent second closest to the ball.

In the second embodiment, the control target region is switched in overall and fixed units according to the estimation value related to the overall action policy. However, the present invention is not limited to this. The switching of the control target region may be applied to switching of a hierarchy of action control.

In the embodiments explained above, the respective control target regions are controlled by the BCI signal for the highest-order action control. However, the present invention is not limited to this. A BCI signal used for control may be dynamically switched according to the switching of the control target region. Specifically, for example, if an agent raises the right hand during the walking, a target that should be controlled is changed from a walking action to an arm action and control of the arm action of the agent is performed by using a processing result of a signal detected from the motor area. By changing at any time where attention of BCI operation is directed, as a result, it is possible to perform BCI control with a high degree of freedom. In this case, concerning the walking action, it is desirable to autonomously perform continuous control by, for example, maintaining the last command.

In the embodiments explained above, the action mode is switched to the autonomous control mode, for example, when a BCI signal is not obtained for the fixed time. However, the present invention is not limited to this. An effective ratio of the autonomous control mode and the BCI control of the agent may be changed according to a percentage of correct answers in the prior processing. In this case, it is possible to control the agent with the BCI signal at a fixed contribution ratio by controlling control content with low learning effect mainly in the autonomous control mode. When the user becomes well-trained, it is possible to control the agent only with the BCI control.

In the first embodiment, the human-type agent built in the inside of the computer is controlled. However, the present invention is not limited to this and can be more widely applied, for example, when a human-type agent as an actual robot is controlled and when various single agents are controlled.

In the second embodiment, the multi-agent in the soccer game is controlled. However, the present invention is not limited to this and can be widely applied to various kinds of control.

In the embodiments explained above, a brain activity signal by the BCI is processed to control the control target. However, the present invention is not limited to this and can be widely applied when a brain activity signal is detected by the BMI.

The present invention can be applied to, for example, various kinds of control by a brain computer interface (BCI) and various kinds of control by a brain machine interface (BMI).

The present application contains subject matter related to that disclosed in Japanese Priority Patent Application JP filed in the Japan Patent Office on Apr. 24, 2008, the entire contents of which is hereby incorporated by reference.

It should be understood by those skilled in the art that various modifications, combinations, sub-combinations, and alterations may occur depending on design requirements and other factors insofar as they are within the scope of the appended claims or the equivalents thereof.

What is claimed is:

1. A control apparatus comprising:
a brain-activity-signal processing unit that processes plural brain activity signals and detects plural estimation values for estimating brain activity; and
a control unit that controls a control target on the basis of the estimation values of the brain activity, wherein
the control unit switches a control target region of the control target according to at least one of the plural brain activity signals and controls the switched control target region according to the plural brain activity signals.

2. A control apparatus according to claim 1, wherein the control target is a single agent.

3. A control apparatus according to claim 1, wherein the control apparatus switches a control mode of the control target to an autonomous control mode when a significant one of the estimation values is not obtained for a fixed time or longer.

4. A control apparatus according to claim 1, wherein the control target is a multi-agent.

5. A control apparatus according to claim 1, wherein the switching of the control target region is switching of a control target region, which is controlled according to an estimation value related to the switching of the control target region, and a control target region lower in order than the control target region, which is controlled according to the estimation value.

6. A control method comprising the steps of:
processing plural brain activity signals and detecting plural estimation values for estimating brain activity; and
controlling a control target on the basis of the estimation values of the brain activity, wherein the control step includes the steps of:
switching a control target region of the control target according to at least one of the plural brain activity signals; and
controlling the switched control target region according to the plural brain activity signals.

7. A computer program for a control method executable by a computer, the computer program comprising the steps of:
processing plural brain activity signals and detecting plural estimation values for estimating brain activity; and
controlling a control target on the basis of the estimation values of the brain activity, wherein
the control step includes the steps of:
switching a control target region of the control target according to at least one of the plural brain activity signals; and
controlling the switched control target region according to the plural brain activity signals.

8. A recording medium having recorded therein a computer program for a control method executable by a computer, the computer program for a control method comprising the steps of:
processing plural brain activity signals and detecting plural estimation values for estimating brain activity; and
controlling a control target on the basis of the estimation values of the brain activity, wherein the control step includes the steps of:
switching a control target region of the control target according to at least one of the plural brain activity signals; and
controlling the switched control target region according to the plural brain activity signals.
